(19) United States
(12) Patent Application Publication
(10) Pub. No.: US 2011/0276110 A1
(43) Pub. Date: Nov. 10, 2011

(54) Power Circuitry for an Implantable Medical Device Using a DC-DC Converter

(75) Inventors: Todd Whitehurst, New York, NY (US); Rafael Carbunaru, Valley Village, CA (US); Jordi Parramon, Valencia, CA (US)

(73) Assignee: Boston Scientific Neuromodulation Corporation, Valencia, CA (US)

(21) Appl. No.: 13/086,549

(22) Filed: Apr. 14, 2011

Related U.S. Application Data
(60) Provisional application No. 61/332,549, filed on May 7, 2010.

(51) Int. Cl. A61N 1/36

(52) U.S. Cl. 607/60; 607/61

(57) ABSTRACT

Improved power circuitry for charging a battery in an implantable medical device is disclosed. The improved power circuitry uses a DC-DC converter positioned between the rectifier and the battery in the implant to be charged, and operates to boost the voltage produced by the rectifier to a higher compliance voltage used to charge the battery. Because the rectifier can now produce a smaller DC voltage, the AC voltage preceding the rectifier (the coil voltage), can also be lessened. Lowering the coil voltage reduces the amount of heat generated by the coil, which reduces the overall heat generated by the implant during receipt of a magnetic charging field from an external charger during a charging session, which improves patient safety. Additionally, a reduced coil voltage means that the external charger can reduce the intensity of the magnetic charging field, which also reduces heat generated in the external charger during the charging session.

### CROSS REFERENCE TO RELATED APPLICATIONS

This is a non-provisional application of U.S. patent application Ser. No. 61/332,549, filed May 7, 2010, to which priority is claimed, and which is incorporated herein by reference in its entirety.

### FIELD OF THE INVENTION

The present invention relates generally to improved power circuitry in an implantable medical device for wirelessly receiving and rectifying power from an external charger for charging a battery in the implantable medical device, in which the power circuitry includes DC-DC conversion circuitry.

### BACKGROUND

Implantable stimulation devices generate and deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, occipital nerve stimulators to treat migraine headaches, and other neural stimulators to treat urinary incontinence, sleep apnea, shoulder sublaxation, etc. The present invention may find applicability in all such applications and in other implantable medical device systems, although the description that follows will generally focus on the use of the invention in a Bion® microstimulator device system of the type disclosed in U.S. Published Patent Application No. 2010/0268309.

Microstimulator devices typically comprise a small generally-cylindrical housing which carries electrodes for producing a desired stimulation current. Devices of this type are implanted proximate to the target tissue to allow the stimulation current to stimulate the target tissue to provide therapy for a wide variety of conditions and disorders. A microstimulator usually includes or carries stimulating electrodes intended to contact the patient's tissue, but may also have electrodes coupled to the body of the device via a lead or leads. A microstimulator may have two or more electrodes. Microstimulators benefit from simplicity. Because of their small size, the microstimulator can be directly implanted at a site requiring patient therapy.

FIG. 1 illustrates an exemplary implantable microstimulator 100. As shown, the microstimulator 100 includes a power source 145 such as a battery, a programmable memory 146, electrical circuitry 144, and a coil 147. These components are housed within a capsule 180, which is usually a thin, elongated cylinder, but may also be any other shape as determined by the structure of the desired target tissue, the method of implantation, the size and location of the power source 145 and/or the number and arrangement of external electrodes 142. In some embodiments, the volume of the capsule 180 is substantially equal to or less than three cubic centimeters.

The battery 145 supplies power to the various components within the microstimulator 100, such the electrical circuitry 144 and the coil 147. The battery 145 also provides power for therapeutic stimulation current sourced or sunk from the electrodes 142. The power source 145 may be a primary battery, a rechargeable battery, a capacitor, or any other suitable power source. Methods for charging battery 145 will be described further below.

The coil 147 is configured to receive and/or emit a magnetic field that is used to communicate with, or receive power from, one or more external devices that support the microstimulator 100, examples of which will be described below. Such communication and/or power transfer may be transcutaneous as is well known.

The programmable memory 146 is used at least in part for storing one or more sets of data, including electrical stimulation parameters that are safe and efficacious for a particular medical condition and/or for a particular patient. Electrical stimulation parameters control various parameters of the stimulation current applied to a target tissue including, but not limited to, the frequency, pulse width, amplitude, burst pattern (e.g., burst on time and burst off time), duty cycle or burst repeat interval, ramp on time and ramp off time of the stimulation current, etc.

The illustrated microstimulator 100 includes electrodes 142-1 and 142-2 on the exterior of the capsule 180. The electrodes 142 may be disposed at either end of the capsule 180 as illustrated, or placed along the length of the capsule. There may also be more than two electrodes arranged in an array along the length of the capsule. One of the electrodes 142 may be designated as a stimulating electrode, with the other acting as an indifferent electrode (reference node) used to complete a stimulation circuit, producing monopolar stimulation. Or, one electrode may act as a cathode while the other acts as an anode, producing bipolar stimulation. Electrodes 142 may alternatively be located at the ends of short, flexible leads. The use of such leads permits, among other things, electrical stimulation to be directed to targeted tissue(s) a short distance from the surgical fixation of the bulk of the device 100.

The electrical circuitry 144 produces the electrical stimulation pulses that are delivered to the target nerve via the electrodes 142. The electrical circuitry 144 may include one or more microprocessors or microcontrollers configured to decode stimulation parameters from memory 146 and generate the corresponding stimulation pulses. The electrical circuitry 144 will generally also include other circuitry such as the current source circuitry, the transmission and receiver circuitry coupled to coil 147, electrode output capacitors, etc.

The external surfaces of the microstimulator 100 are preferably composed of biocompatible materials. For example, the capsule 180 may be made of glass, ceramic, metal, or any other material that provides a hermetic package that excludes water but permits passage of the magnetic fields used to transmit data and/or power. The electrodes 142 may be made of a noble or refractory metal or compound, such as platinum, iridium, tantalum, titanium, titanium nitride, niobium or alloys of any of these, to avoid corrosion or electrolysis which could damage the surrounding tissues and the device.

The microstimulator 100 may also include one or more infusion outlets 182, which facilitate the infusion of one or more drugs into the target tissue. Alternatively, catheters may be coupled to the infusion outlets 182 to deliver the drug therapy to target tissue some distance from the body of the microstimulator 100. If the microstimulator 100 is configured to provide a drug stimulation using infusion outlets 182, the microstimulator 100 may also include a pump 149 that is configured to store and dispense the one or more drugs.

Turning to FIG. 2, the microstimulator 100 is illustrated as implanted in a patient 150, and further shown are various external components that may be used to support the implanted microstimulator 100. An external controller 155 may be used to program and test the microstimulator 100 via communication link 156. Such link 156 is generally a two-way link, such that the microstimulator 100 can report its status or various other parameters to the external controller 155. Communication link 156 is established via magnetic inductive coupling. Thus, when data is to be sent from the external controller 155 to the microstimulator 100, a coil 158 in the external controller 155 is excited to produce a magnetic field that comprises the link 156, which magnetic field is detected at the coil 147 in the microstimulator. Likewise, when data is to be sent from the microstimulator 100 to the external controller 155, the coil 147 is excited to produce a magnetic field that comprises the link 156, which magnetic field is detected at the coil 158 in the external controller. Typically, the magnetic field is modulated, for example with Frequency Shift Keying (FSK) modulation or the like, to encode the data.

An external charger 151 provides power used to recharge the battery 145 (FIG. 1). Such power transfer occurs by energizing the coil 157 in the external charger 151, which produces a magnetic charging field comprising link 152. This magnetic charging field 152 energizes the coil 147 through the patient 150's tissue, and which is rectified, filtered, and used to recharge the battery 145 as explained further below. Link 152, like link 156, can be bidirectional to allow the microstimulator 100 to report status information back to the external charger 151. For example, once the circuitry 144 in the microstimulator 100 detects that the power source 145 is fully charged, the coil 147 can signal that fact back to the external charger 151 so that charging can cease. Charging can occur at convenient intervals for the patient 150, such as every night.

FIG. 3 illustrates salient portions of the microstimulator's power circuitry 160. Charging energy (i.e., the magnetic charging field) is received at coil 147 via link 152. The coil 147 in combination with capacitor 162 comprises a resonant circuit, or tank circuit, which produces an AC voltage, Va, which is generally characterized by its rms value. This AC voltage is rectified by rectifier circuitry 164 to produce a DC compliance voltage Vb used in charging the battery 145. Rectifier circuitry 164 can comprise a full-or half-wave rectifier, although it is shown in FIG. 3 as a single diode for simplicity. Capacitor 166 assists to filter Vb, although Vb may have a negligible ripple not requiring filtering. The compliance voltage Vb is received by charging circuitry 170, a type of conditioning circuitry which ultimately takes the DC voltage Vb and uses it to produce a controlled battery charging current, Ibat. Charging circuitry 170 is well known. One skilled in the art will recognize that the power circuitry 160 may include other components not shown for simplicity.

It is generally desirable to charge the battery 145 as quickly as possible to minimize inconvenience to the patient. One way to decrease charging time is to increase the intensity of the magnetic charging field by increasing the excitation current, Iprim, in the coil 157 of the external charger. Increasing the magnetic charging field will increase the current/voltage induced in the coil 147 of the microstimulator 100, which increases the battery charging current, Ibat.

However, the intensity of the magnetic charging field can only be increased so far before implant heating becomes a concern. One skilled in the art will understand that implant heating is an inevitable side effect of charging using magnetic fields. Heating can result from several different sources, such as eddy currents in conductive portions of the implant, or heating of the various components in the power circuitry 160. One large contributor to heat generation in the microstimulator 100 is the coil 147. Implant heating is a serious safety concern: if an implant exceeds a given safe temperature (e.g., 41° C.), the tissue surrounding the implant may be aggravated or damaged, injuring the patient.

The art has recognized that implant heating can be controlled by controlling the intensity of the magnetic charging field produced at the external charger 151. For example, the excitation current, Iprim, flowing through charging coil 157 can be reduced to reduce the temperature of the implant during a charging session. The art has also recognized that implant heating can be regulated by duty cycling the charging field, i.e., by turning the charging field at the external charger 151 on and off. See, e.g., U.S. patent application Ser. No. 12/575,733, filed Oct. 8, 2009, which is incorporated herein by reference.

While changing the intensity or duty cycling of the magnetic charging field produced by the external charger 151 can be an effective means of controlling implant temperature, the inventors have realized that other improvements, discussed further, can also remedy this problem.

### BRIEF DESCRIPTION OF THE DRAWINGS

FIG. 1 illustrates a microstimulator implant, including a battery requiring periodical recharging from an external charger, in accordance with the prior art.

FIG. 2 shows the implant in communication with, inter alia, an external charger in accordance with the prior art.

FIG. 3 illustrates power circuitry within the implant in accordance with the prior art.

FIGS. 4A and 4B illustrate a simulation of operation of the external charger and power circuitry in accordance with the prior art.

FIG. 5 illustrates improved power circuitry for an implant in accordance with an embodiment of the invention, which circuitry includes a DC-DC converter between a rectifier and the battery.

FIG. 6 illustrates one example of a DC-DC converter for use in the improved power circuitry.

FIGS. 7A and 7B illustrate alternative DC-DC converters that can be used in the improved power circuitry.

FIGS. 8A and 8B respectively illustrate the difference in operation between the improved power circuitry and the power circuitry of the prior art.

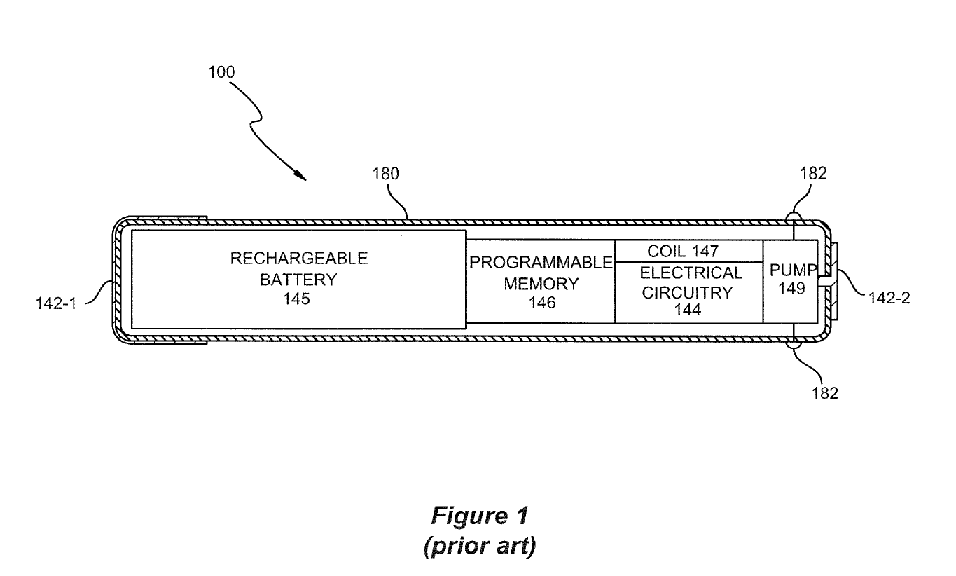

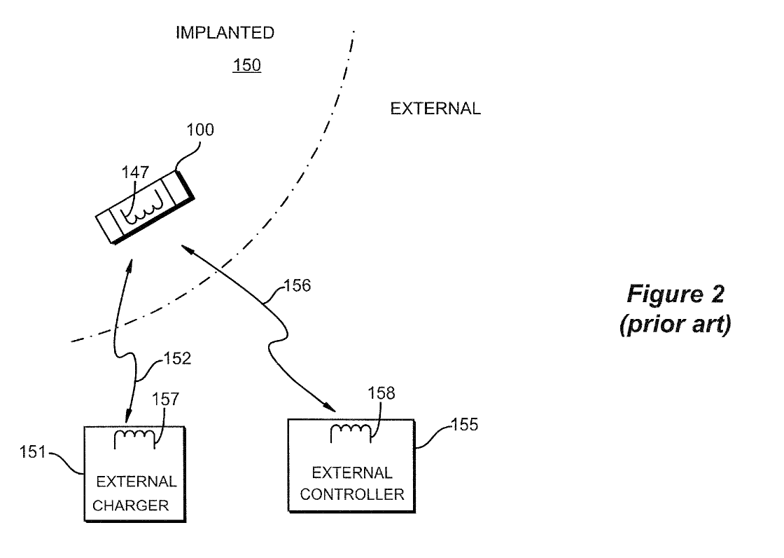

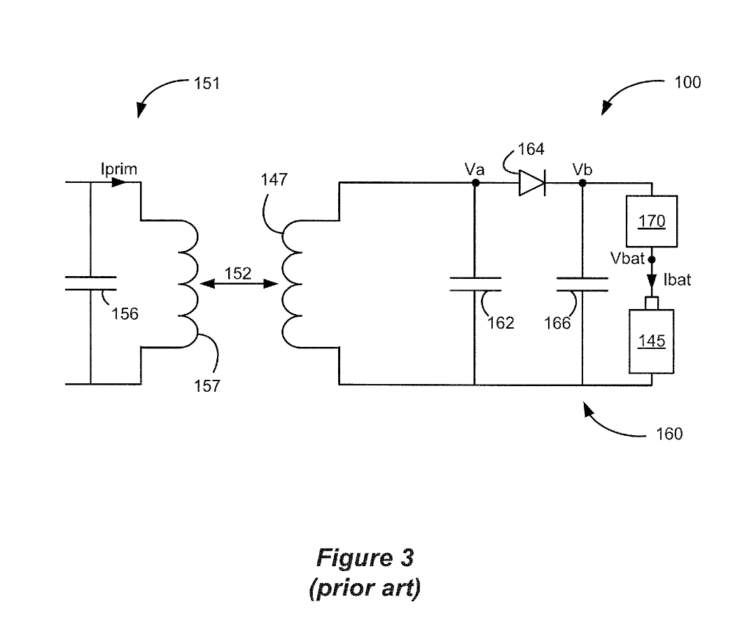

### DETAILED DESCRIPTION

Improved power circuitry for charging a battery in an implantable medical device is disclosed. The improved power circuitry uses a DC-DC converter positioned between the rectifier and the battery in the implant to be charged, and operates to boost the voltage produced by the rectifier to a higher compliance voltage used to charge the battery. Because the rectifier can now produce a smaller DC voltage, the AC voltage preceding the rectifier (the coil voltage), can also be lessened. Lowering the coil voltage reduces the amount of heat generated by the coil, which reduces the overall heat generated by the implant during receipt of a magnetic charging field from an external charger during a charging session, which improves patient safety. Additionally, a reduced coil voltage means that the external charger can reduce the intensity of the magnetic charging field, which also reduces heat generated in the external charger during the charging session.

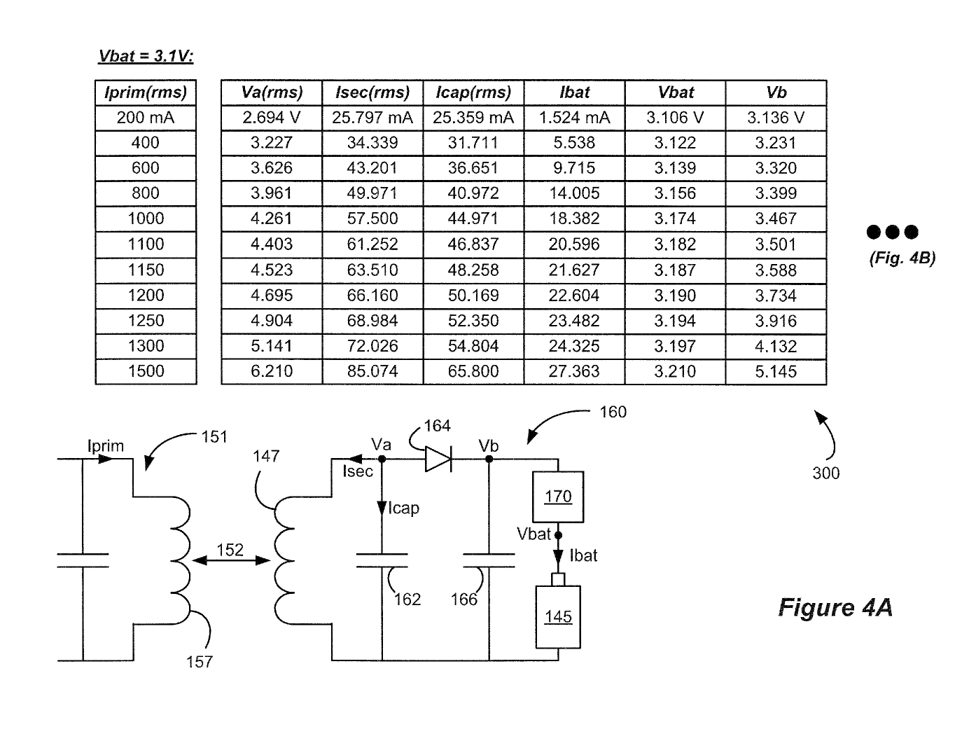

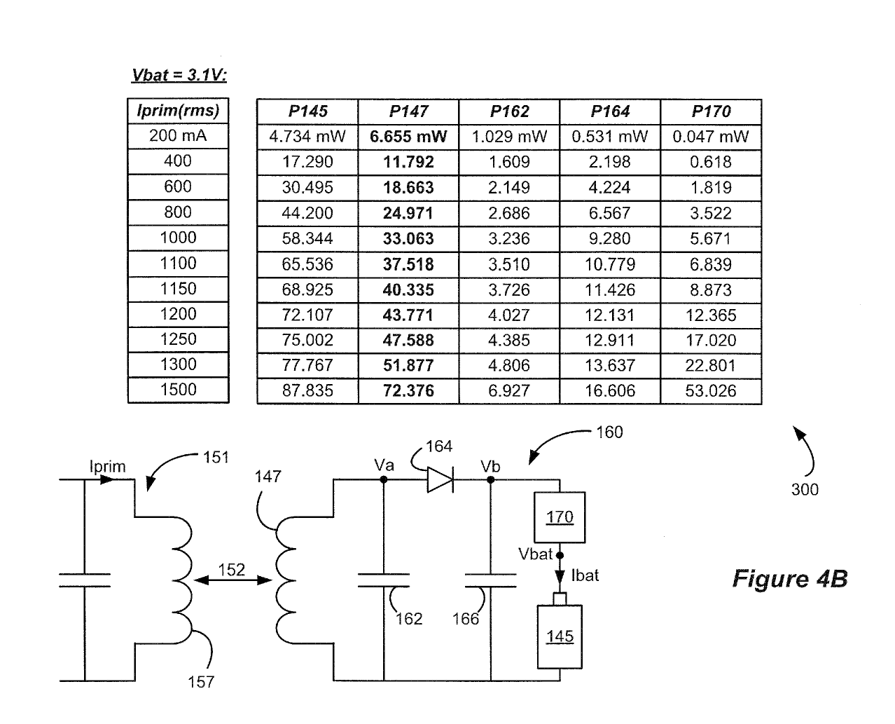

FIGS. 4A and 4B show simulated results 300 for the power dissipated in the power circuitry 160 of a prior art microstimulator 100 during a charging session, which simulation is also discussed in the above-incorporated '733 application. Simulation 300 shows the effect of varying the current in the external controller's charging coil 157 (Iprim(rms)) (and thus the intensity of the resulting magnetic charging field) on the various components in the power circuitry 160 of the implant 100, with each successive row representing an increasing value for Iprim(rms). Because the simulation 300 will vary depending on how full or depleted the implant battery 145 is at a given moment, the depicted simulation assumes a battery with a particular voltage of Vbat=3.1 V.

Simulation 300 assumes a particular coupling factor between the primary coil 157 in the external charger 151 and the secondary coil 147 in the implant 100, which coupling factor is modeled taking into account factors affecting such coupling, such as coil inductances, coil alignment, the distance and permittivity of any materials (e.g., tissue, air) between the coils, etc. Once a coupling factor is chosen, and as shown in FIG. 4A, simulated induced currents and voltages in the power circuitry 160 can be determined, including the current in charging coil 147 (Isec(rms)), the current in the associated tank capacitor 162 (Icap(rms)), the voltage across the coil 147 (Va(rms)), the DC compliance voltage produced by the rectifier circuit (diode) 164 (Vb), the battery charging current (Ibat), the battery voltage (Vbat) resulting from the input of the battery charging current, which battery voltage takes into account the internal resistance of the battery 145. Of course, relevant parameters for the various components in the power circuitry 160 (resistances, capacitance, inductances, coupling factor, etc.) are input into the simulation program to allow it to generate the simulation results.

From the various simulated voltages and currents in FIG. 4A, the simulation 300 can further calculate the power dissipated by the various components in the power circuitry 160, as shown in FIG. 4B, which powers essentially comprise the product of the voltage across and current through the various components. As shown, the power drawn by each component is represented by the element numeral for the component: for example, the power drawn by the battery 145 during charging is denoted as P145. Note the power drawn by the coil 147 (P147), bolded for easy viewing, is significant. It is proportional to the square of the rms voltage across the coil 147 (Va(rms)) and the intrinsic resistance of the coil.

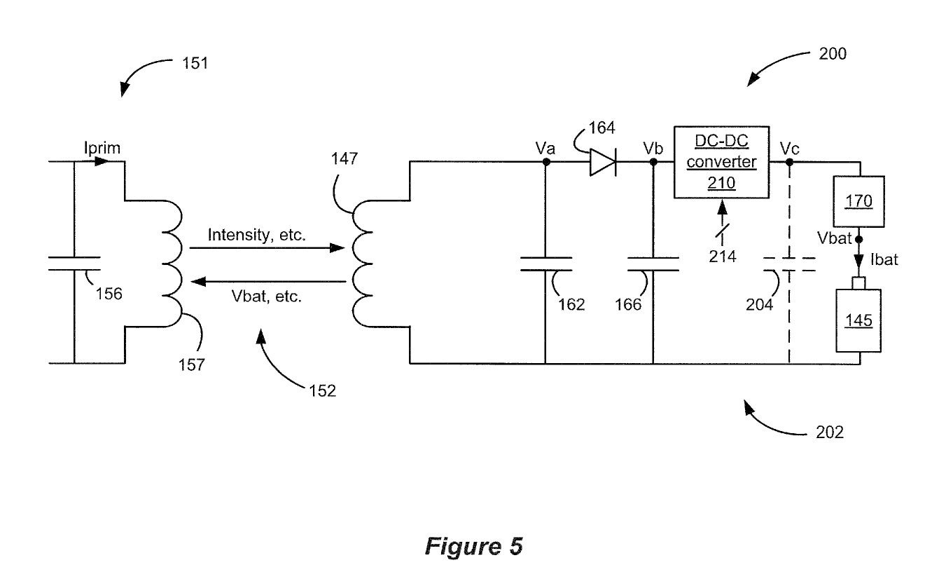

One goal of the present invention is to reduce the power dissipated by the coil 147 during a charging session to improve patient safety, and FIG. 5 illustrates improved power circuitry 202 for an improved microstimulator 200 that achieves that result. Because most of the components in improved power circuitry 202 have already been discussed, the functions of such components are not reiterated. However, new to improved power circuitry 202 is the inclusion of a DC-DC converter 210, which intervenes between the rectifier 164 and the charging circuitry 170. As in the prior art, Vb comprises a DC voltage produced by rectifier 164, which again can comprise a full- or half-wave rectifier for example. DC voltage Vb is input to DC-DC converter 210, which in turn produces a new higher DC compliance voltage, Vc. Vc is input into the charging circuitry 170 to produce a controlled battery charging current, Ibat.

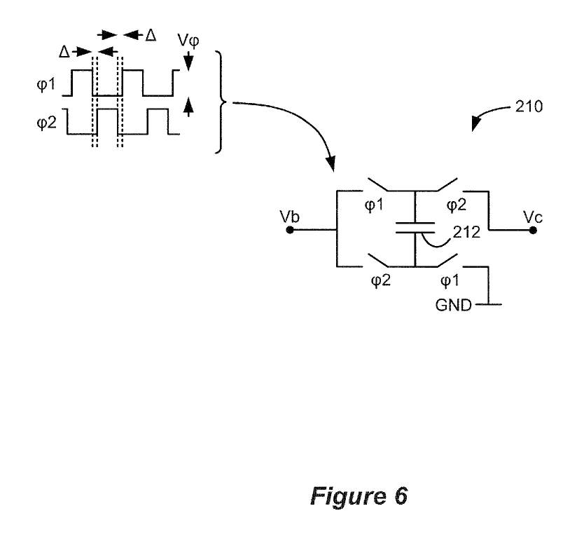

As just noted, DC-DC converter 210 boosts voltage Vb to a higher voltage Vc, and in one embodiment, DC-DC converter 210 can comprise a charge pump. More specifically, the charge pump can comprise a “doubler,” thereby producing a voltage Vc that is twice the voltage of Vb (i.e., Vc=2 Vb, a scalar of 2). An example of such a doubler is shown in FIG. 6. In the embodiment shown, two clocks, 100 1 and φ2, govern the charging of a capacitor 212. The clocks φ1 and φ2, as the timing diagram in FIG. 6 suggests, are essentially 50% duty cycle clocks which are out of phase, although as shown, each of the clocks are off together for a brief period, Δ, to prevent interference between the two clock phases. In operation, during the assertion of φ1, the capacitor 212 is charged to voltage Vb. Then, during the assertion of φ2, the previously-grounded (lower) plate of capacitor 212 is coupled to the intermediate voltage Vb. Because Vb was already present on the top plate of the capacitor 212, presenting that same voltage to the bottom plate boosts the voltage on the top plate to roughly 2 Vb, which is output as Vc. An optional filtering capacitor 204 (FIG. 5) helps to stabilize Vc produced by the DC-DC converter 210, but is not strictly necessary, particularly if the charging circuitry 170 is capable of handling slight variations in the compliance voltage, Vc. Capacitor 212 should be large enough to supply sufficient power to the charging circuitry 170. Capacitor 212 may comprise a discrete component within the microstimulator 200, or may reside on the analog integrated circuitry otherwise typically present within the microstimulator.

A doubler is preferred for the DC-DC converter 210 for its simplicity: it requires only a minimal number of components and is easily controlled by clock signal ultimately issuing from a microcontroller (not shown) already present in the microstimulator 200. Moreover, a capacitor-based charge pump provides a particularly good design because it is efficient and doesn't draw significant amounts of power, leaving more power available for charging the battery 145. Additionally, low power draw in the DC-DC converter 210 means that such circuitry will not contribute significantly to heat generation in the microstimulator 200.

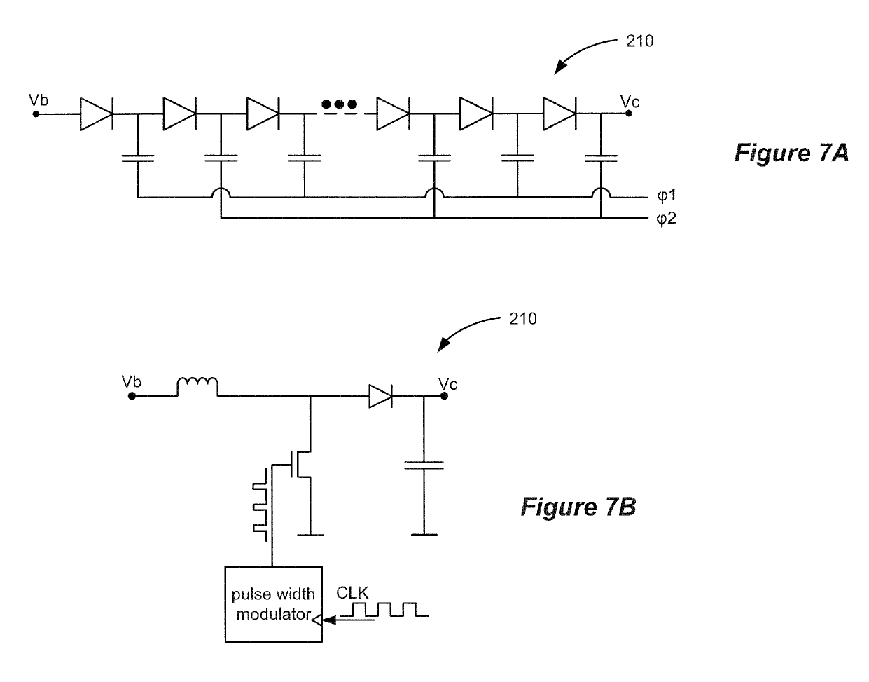

Other types of DC-DC converters 210 could be used in improved power circuitry 202, and in particular converters can be used which allow the scalar of the DC-DC converter to be adjusted. For example, a staged charge pump as shown in FIG. 7A can also be used. As is well known, this arrangement allows the compliance voltage, Vc, to be set in accordance with the number of diode-capacitor stages used, i.e., Vc=Vb+N(Vφ−Vd)−Vd, where Vd equals the voltage drop across one of the diodes, Vφ equals that magnitude of the clocking signals (see FIG. 6), and N equals the number of stages in the bank. Therefore, by controlling either N or Vφ, the magnitude of Vc can be set to an appropriate value. For example, switches (not shown) could be provided to bypass any of the N stages in the capacitor-diode bank 240 to set Vc appropriately. Such adjustments to Vc can be made via optional control signal(s) 214, as shown in FIG. 5, which control signal(s) 214 ultimately issue from the microstimulator 200's microcontroller.

Optionally, a boost converter can be used for DC-DC converter 210, as shown in FIG. 7B. Because the circuitry and operation of a boost converter is well known, and is explained in U.S. Published Patent Application No. 2010/0211132, which is hereby incorporated by reference in its entirety, such circuitry is not explained in depth here. However, because a boost converter includes a current-drawing inductor capable of generating heat, it may in some cases be counter-indicated for use as the DC-DC converter 210 for improved power circuitry 202, although in other cases use of a boost converter may be fine, particular if the boost converter is only used to provide a small boost to Vb (Vc=n*Vb, where scalar n is relatively small). Once again, the scalar provided by the boost converter is controllable, and may once again be generally indicated via control signal(s) 214 in FIG. 5.

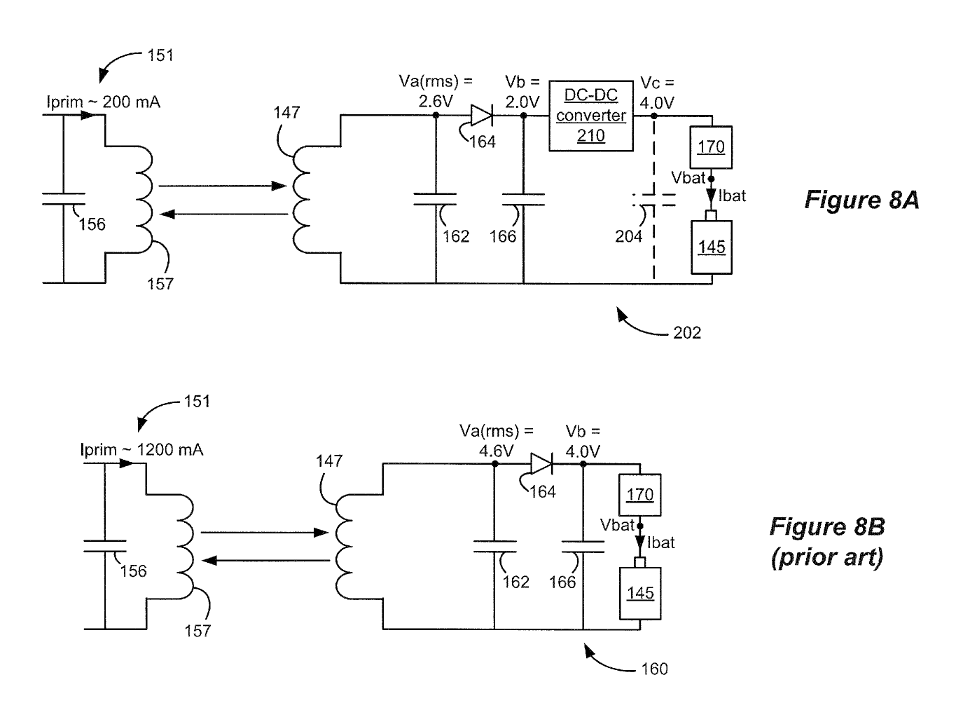

Some of the benefits of the improved power circuitry 202 when compared to power circuitry 160 of the prior art are illustrated in FIG. 8A and 8B respectively. In both cases, it is assumed that a compliance voltage of 4.0V is needed to charge the battery 145 in the microstimulator, and so that voltage is applied by both circuits to the charge circuitry 170, i.e., Vc=4.0V in FIG. 8A and Vb=4.0 in FIG. 8B. Assuming a doubler is used for DC-DC converter 210 in FIG. 8A, this means a voltage of half this amount, 2.0 V, is needed at the input to the converter 210, i.e., Vb=2.0 V. Continuing to work backwards in power circuitry 202 of FIG. 8A, an rms coil voltage Va(rms) that is slightly higher will be required to generate Vb. For example, and assuming a half-wave rectifier 164 is used to generate Vb=2.0V, a rms voltage Va(rms)=2.6V is needed, as approximately 0.6 V will be lost across the diode due to its threshold voltage (Vd=0.6). (In other words, Va(rms)˜Vd+Vb). (If a full-wave rectifier is used, the loss across the rectifier 164 would be doubled to 1.2V, requiring Va(rms) to be 3.2V). By comparison, in the prior art power circuitry 160 of FIG. 8B, which lacks a DC-DC converter, a rms voltage Va(rms)=4.6V is required to generate the Vb=4.0V compliance voltage, which difference is again due to the loss across the rectifier 164.

To summarize, to create the same compliance voltage of 4.0V in either of the power circuitries of FIGS. 8A and 8B, differing coil voltages Va(rms) are required: 2.6V versus 4.6V. Several benefits flow from the reduction of this coil voltage, Va(rms), in the improved power circuitry 202. First, a lower coil voltage will generate less current in the coil 147, and therefore generate less heat in the microstimulator 200. Because the power drawn by the coil 147 scales with the square of Va(rms), reduction of Va(rms) provides power savings that are that much more significant. As noted earlier, reducing the heat generation in the implant is a significant benefit, particularly as concerns the safety of the microstimulator.

Additionally, it is easier for the external charger 151 to induce a lower coil voltage, and therefore the intensity of the magnetic charging field generated by the external charger can be lessened. As noted earlier, the intensity of the generated magnetic charging field scales with the magnitude of the excitation current, Iprim, in the coil 157 of the external charger. Therefore, a lower coil voltage Va(rms) requires a smaller excitation current, Iprim, a fact which can be noticed in the simulation 300 of FIG. 4A. As the simulation 300 shows, generating a coil voltage Va(rms) of 2.6V—as indicated for the improved power circuitry 202 of FIG. 8A—requires an excitation current Iprim equal to approximately 200 mA. By contrast, generating a coil voltage Va(rms) of 4.6V—as indicated for the prior art power circuitry 202 of FIG. 8B—requires an excitation current Iprim equal to approximately 1200 mA. The result is that the external charger 151 need not expend as much energy when charging a microstimulator 200 having the improved power circuitry 202 of FIG. 8A.

Lessening the power requirements at the external charger 151 has its own benefits. The external charger 151 is, like the microstimulator 200, susceptible to heating, in particular due to eddy currents induced by the magnetic charging field in the charger's conductive components. See, e.g., U.S. patent application Ser. No. 12/689,392, filed Jan. 19, 2010, discussing external charger heating in further detail. Additionally, and assuming the external charger 151 is powered by a battery, lessening the power requirements can prolong the life of that battery.

Use of improved power circuitry 202 will not significantly affect traditional methods of communication between the external charger 151 and the microstimulator 200 during a charging session. Referring again to FIG. 5, the external charger 151 provided a magnetic charging field 152a to the receiving coil 147 in the microstimulator 200. This field 152a will be varied in intensity (by varying Iprim), and may also be varied in other manners, such as by varying the duty cycle as explained in the above-incorporated '733application. Such variations can occur based on feedback 152b received from the microstimulator 200. Such feedback 152b can be transmitted from the microstimulator 200 to the external charger 151 by creating data-modulated reflection in the magnetic charging field by varying the impedance of the microstimulator's coil 147. This method of communication is sometimes referred to as Load Shift Keying (LSK) and has been discussed in the art. Feedback 152b can include the capacity of the battery 145 (i.e., Vbat), which among other things can inform the external charger 151 when the battery 145 is full and thus that production of the magnetic charge field can cease. Feedback 152b can include other information as well, such as information indicative of the coupling between the external charger 151 and the microstimulator 200, such as the Vnab parameter (the voltage across the charging circuitry 170) discussed in the above-incorporated '733 application. Such information can be useful to the external charger in setting the intensity and duty cycle of the magnetic charging field, again as explained in the '733 application. Note that optimization of the magnetic charging field by the external charger based on received feedback 152b may require adjustment given the addition of the DC-DC converter 210 in the improved power circuitry 202. For example, new simulations 300 (FIGS. 4A and 4B) may be needed to provide a basis for allowing the external charger to properly understand the operation of the power circuitry 202 in light of the boosted voltages provided by the DC-DC converter 210.

As discussed to this point, the compliance voltage Vc generated by the DC-DC converter 210 is supplied to charging circuitry 170. Charging circuitry 170 can be useful in power circuitry 202 because it can regulate the incoming compliance voltage Vc to provide a controlled charging current (or voltage) for the battery 145. Charging circuitry 170 can further be useful because of its ability to report to and receive control from the implant's microcontroller, which communication can be used to establish a charging scheme such as one that varies the battery charging current, Ibat, over time during the charging session. However, use of charging circuitry 170, while beneficial, is not strictly necessary in embodiments of the disclosed invention. For example, the compliance voltage Vc generated by the DC-DC converter 210 can be supplied directly to the battery 145 to charge it. Or, that compliance voltage Vc can be processed by some other conditioning or buffering circuitry (collectively, “conditioning circuitry”) before being presented to the battery 145. Thus, in embodiments of the invention, the compliance voltage Vc is used as the power to directly or indirectly charge the battery 145, and in either case the benefits of a lower coil voltage, Va(rms), are preserved.

The foregoing description related to use of an improved power circuitry in a microstimulator. However, it is to be understood that the invention is not so limited, and could be used with any type of implantable medical device. For example, the present invention may be used in an implantable sensor, an implantable pump, a pacemaker, a defibrillator, a cochlear stimulator, a retinal stimulator, a spinal cord stimulator, a stimulator configured to produce coordinated limb movement, a cortical and deep brain stimulator, or with any other neural stimulator configured to treat any of a variety of conditions.

While the inventions disclosed have been described by means of specific embodiments and applications thereof, numerous modifications and variations could be made thereto by those skilled in the art without departing from the literal and equivalent scope of the inventions set forth in the claims.

1. An implantable medical device, comprising:
a coil for receiving a magnetic charging field from an external charger, the magnetic charging field inducing an AC voltage in the coil;
a rectifier for producing a first DC voltage from the AC voltage;
a converter for producing a second DC voltage from the first DC voltage, wherein the second DC voltage is higher than the first DC voltage, and wherein the converter is adjustable to adjust the magnitude of the second DC voltage; and
a battery, wherein the second DC voltage provides power to charge the battery.

2. The device of claim 1, wherein the converter comprises a charge pump.

3. (canceled)

4. The device of claim 1, wherein the converter comprises an inductor.

5. (canceled)

6. The device of claim 1, wherein the second DC voltage provides power directly to the battery to charge the battery.

7. The device of claim 1, wherein the second DC voltage provides power indirectly to the battery through a conditioning circuit which provides a charging current to charge the battery.

8. The device of claim 1, further comprising a conditioning circuit between the converter and the battery.

9. The device of claim 1, wherein the rectifier comprises a half- or full-wave rectifier.

10. The device of claim 1, further comprising at least one electrode coupled to the implantable medical device for providing electrical stimulation to a patient's tissue.

11. The device of claim 1, wherein the coil is further configured to communicate data to the external charger during receipt of the magnetic charging field.

12. An implantable medical device, comprising:
a coil for receiving a magnetic charging field from an external charger, the magnetic charging field inducing an AC voltage in the coil;
a rectifier for producing a first DC voltage from the AC voltage;
a converter for producing a second DC voltage from the first DC voltage, wherein the second DC voltage is higher than the first DC voltage, and wherein the converter is adjustable to adjust the magnitude of the second DC voltage;
a conditioning circuit for receiving the DC voltage and for producing a battery charging current; and
a battery for receiving the battery charging current.

13. The device of claim 12, wherein the converter comprises a charge pump.

14. (canceled)

15. The device of claim 12, wherein the converter comprises an inductor.

16. (canceled)

17. The device of claim 12, wherein the conditioning circuitry implements a charging scheme for varying the battery charging current over time.

18. The device of claim 12, wherein the rectifier comprises a half- or full-wave rectifier.

19. The device of claim 12, further comprising at least one electrode coupled to the implantable medical device for providing electrical stimulation to a patient's tissue.

20. The device of claim 12, wherein the coil is further configured to communicate data to the external charger during receipt of the magnetic charging field.
